(19) United States
(12) Patent Application Publication
(10) Pub. No.: US 2024/0424194 A1
(43) Pub. Date: Dec. 26, 2024

(54) METHOD FOR TREATING RHINOSINUSITIS

(71) Applicant: OLYMPUS MEDICAL SYSTEMS CORP., Tokyo (JP)

(72) Inventor: Daiki ARIYOSHI, Tokyo (JP)

(73) Assignee: OLYMPUS MEDICAL SYSTEMS CORP., Tokyo (JP)

(21) Appl. No.: 18/774,115

(22) Filed: Jul. 16, 2024

Related U.S. Application Data
(63) Continuation-in-part of application No. 18/213,394, filed on Jun. 23, 2023.

(51) Int. Cl. A61M 3/02; A61B 1/00; A61B 1/06; A61B 1/233; A61M 11/02

(52) U.S. Cl. CPC A61M 3/0233; A61B 1/00009; A61B 1/0005; A61B 1/0638; A61B 1/233; A61M 11/02; A61M 2210/0618; A61M 2210/0681

(57) ABSTRACT

A method for treating rhinosinusitis includes inserting a tubular spray device into a target area of the nose and paranasal sinuses, sending a gas into the target area through the spray device to expel deposits from the target area, and spraying a drug into the target area through the spray device.

### TECHNICAL FIELD

The present invention relates to a method for treating rhinosinusitis.

### BACKGROUND ART

Conventionally, various methods for treating rhinosinusitis are known, such as washing with a drug-containing saline solution, nasal sprays, and drug-eluting implants. Methods for treating rhinosinusitis and the devices used for treatment are limited because the nose and paranasal sinuses are small spaces.

### SUMMARY OF THE INVENTION

### Technical Problem

Since the mucosa that develops rhinosinusitis is covered by deposits of pus, mucins and the like, the therapeutic effects of drugs are reduced. For example, if a nasal spray is used, the drug cannot reach the mucosa due to the presence of deposits, and the drug cannot effectively act on the mucosa.

### Solution to Problem

One aspect of the present invention is a method for treating rhinosinusitis, the method including inserting a tubular spray device into a target area of nose and paranasal sinuses, sending a gas into the target area through the spray device to expel deposits from the target area, and spraying a drug into the target area through the spray device.

Another aspect of the present invention is a method for treating rhinosinusitis, the method including inserting a tubular spray device into a target area of nose and paranasal sinuses, and sending a gas and a drug simultaneously into the target area through the spray device to spray the drug while expelling deposits from the target area.

### BRIEF DESCRIPTION OF THE DRAWINGS

FIG. 1 is a configuration diagram of a system used in a method for treating rhinosinusitis.

FIG. 2 is a flowchart of a method for treating rhinosinusitis according to a first embodiment of the present invention.

FIG. 3A is a diagram for explaining the structure of the nasal cavity and the paranasal sinuses as viewed from a side.

FIG. 3B is a diagram for explaining the structure of the nasal cavity and the paranasal sinuses as viewed from the front.

FIG. 4A is a diagram illustrating an example of an endoscopic image in step S1.

FIG. 4B is a diagram illustrating an example of an endoscopic image in step S3.

FIG. 5 is a flowchart of a modification of the method for treating rhinosinusitis in FIG. 2.

FIG. 6 is a flowchart of a method for treating rhinosinusitis according to a second embodiment of the present invention.

FIG. 7 is a diagram illustrating relationships between symptom levels and discrimination colors in a method for treating rhinosinusitis according to a third embodiment of the present invention.

FIG. 8 is a diagram illustrating a relationship between symptom levels and threshold values in a method for treating rhinosinusitis according to the third embodiment of the present invention.

FIG. 9 is a flowchart of a method for treating rhinosinusitis according to the third embodiment of the present invention.

FIG. 10 is a diagram illustrating an example of a white light image of the nose and paranasal sinuses in entirety.

FIG. 11 is a diagram illustrating an example of a discrimination image of the nose and paranasal sinuses in entirety.

FIG. 12 is a diagram illustrating an example of a superimposed image obtained by superimposing a partial discrimination image onto the central portion of a white light image of the nose and paranasal sinuses in entirety.

FIG. 13 is a flowchart illustrating a modification of a method for treating rhinosinusitis according to the third embodiment.

FIG. 14 is a flowchart of a method for treating rhinosinusitis according to a fourth embodiment of the present invention.

FIG. 15 is a flowchart illustrating a modification of a method for treating rhinosinusitis according to the fourth embodiment.

### DESCRIPTION OF EMBODIMENTS

Hereinafter, a method for treating rhinosinusitis according to a first embodiment of the present invention will be described with reference to the drawings.

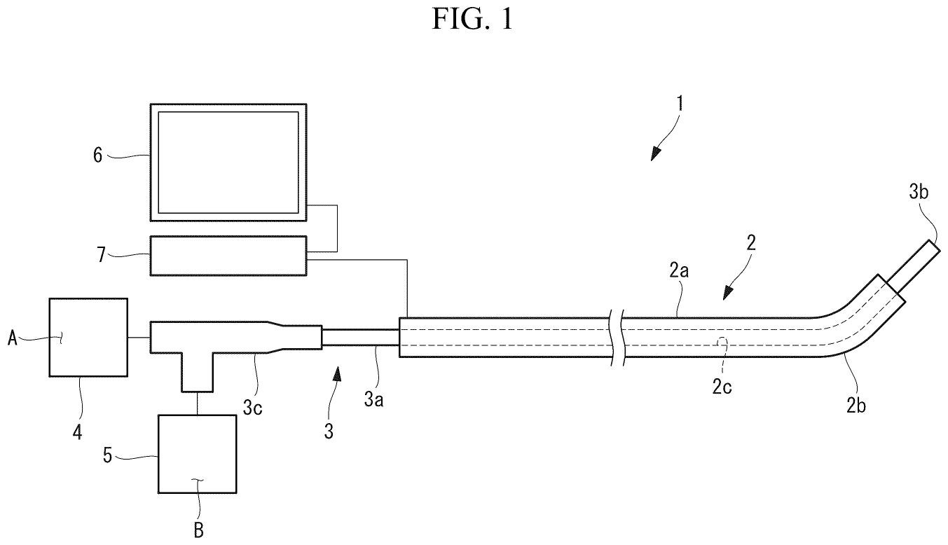

FIG. 1 illustrates an example of a system 1 used in a method 100 for treating rhinosinusitis according to the present embodiment.

The system 1 includes an endoscope 2, a tubular spray device 3, a drug supply source 4 that supplies a drug A to the spray device 3, and a gas supply source 5 that supplies a gas B to the spray device 3.

The endoscope 2 is a flexible scope having an elongated and flexible insertion part 2a. The insertion part 2a has a bendable bending part 2b and a channel 2c running longitudinally through the insertion part 2a. Endoscopic images acquired by the endoscope 2 are displayed on a display 6. The system 1 may further include an image processor 7 that processes endoscopic images.

The spray device 3 is a catheter that can be inserted into the channel 2c. The spray device 3 includes an elongated and flexible tube 3a, a nozzle 3b located at the tip of the tube 3a, and a connector 3c located at the base end of the tube 3a.

The connector 3c is a three-way valve, for example, such that the drug supply source 4 and the gas supply source 5 can be connected to the connector 3c at the same time. Alternatively, the connector 3c may be configured such that the drug supply source 4 and the gas supply source 5 are alternatively connected thereto.

The drug supply source 4 supplies the drug A into the tube 3a through the connector 3c.

The drug supply source 4 is a syringe containing the drug A in liquid form, for example. The supplied drug A is sprayed in a mist from the nozzle 3b.

The drug A may be in powder form. For example, the drug supply source 4 may be a syringe containing the drug A in powder form and a gas such as air, and may supply the drug A and the gas simultaneously into the tube 3a. Otherwise, the drug supply source 4 may supply the drug A in powder form into the tube 3a and subsequently supply the gas into the tube 3a.

The gas supply source 5 supplies air B into the tube 3a through the connector 3c. The gas supply source 5 is a syringe containing the air B, for example. The supplied air B spurts out from the nozzle 3b.

The drug supply source 4 and the gas supply source 5 may be any other devices capable of supplying the drug A and the air B, respectively, into the tube 3a, and each may include a pump to send the drug A or the air B, for example.

The drug A contains an active ingredient that has a therapeutic effect on rhinosinusitis. The active ingredient is selected from among anti-inflammatory agents such as steroids, antibiotics, antivirals, antileukotrienes, molecularly targeted agents, antiallergic agents, antifungal agents, and physiological saline solutions, for example.

The drug A in liquid form preferably has a high viscosity. For example, an additive for increasing viscosity may be pre-added to the drug A in liquid form, or the additive may be supplied to the tube 3a at the same time as the drug A. With a high viscosity, the drug A can remain on the mucosa for a long time, thereby exhibiting a higher therapeutic effect. With a low viscosity, the drug A in liquid form easily flows away from the mucosa and does not readily remain on the mucosa for a long time.

Next, the method 100 for treating rhinosinusitis according to the present embodiment will be described.

The present embodiment illustrates a case in which the spray device 3 is inserted through the channel 2c of the flexible endoscope 2 up to an opening D to a target area C of the nose and paranasal sinuses or the vicinity thereof. However, alternatively, the target area C of the nose and paranasal sinuses may be observed with a rigid scope as the spray device 3 is inserted through the inside of a catheter inserted separately from the rigid scope.

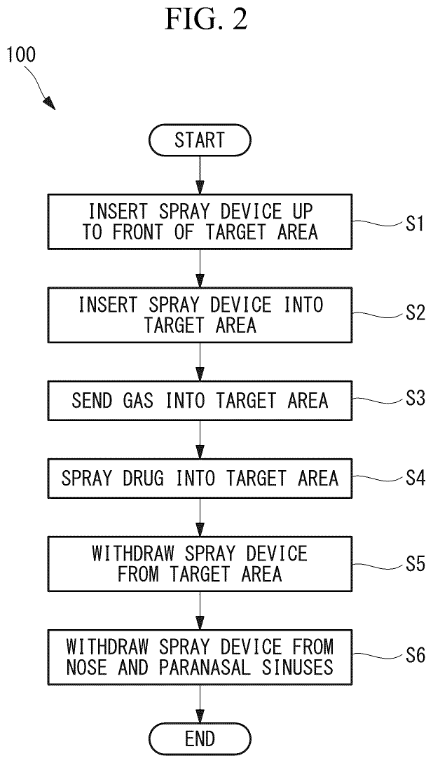

As illustrated in FIG. 2, the method 100 includes a step S1 for inserting the spray device 3 up to the opening D to the target area C of the nose and paranasal sinuses or the vicinity thereof, a step S2 for inserting the spray device 3 into the target area C from the opening D, a step S3 for sending the air B into the target area C through the spray device 3, a step S4 for spraying the drug A into the target area C through the spray device 3, a step S5 for withdrawing the spray device 3 from the target area C, and a step S6 for withdrawing the spray device 3 from the nose and paranasal sinuses.

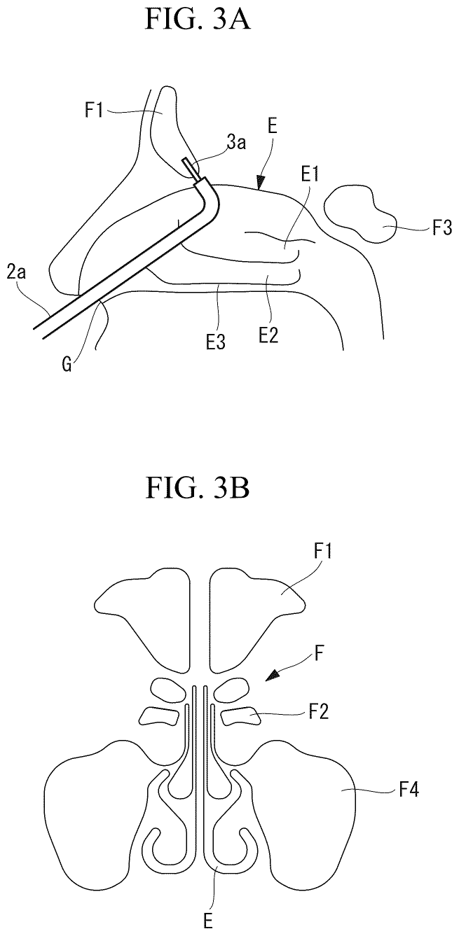

As illustrated in FIGS. 3A and 3B, the nose and paranasal sinuses include a nasal cavity E and paranasal sinuses F. The nasal cavity E has a plurality of areas, including an upper nasal passage E1, a middle nasal passage E2, and a lower nasal passage E3. The paranasal sinuses F have a plurality of areas, including frontal sinuses F1, ethmoid sinuses F2, sphenoid sinuses F3, and a maxillary sinuses F4. The target area C is any of these areas of the nasal cavity E and the paranasal sinuses F.

In this method 100, a first operator operates the endoscope 2, and a second operator different from the first operator operates the spray device 3. The first operator is a doctor, for example, and the second operator is a nurse, for example.

In step S1, the spray device 3 is made to approach up to the opening D to the target area C or the vicinity thereof while the interior of the nose and paranasal sinuses is observed with the endoscope 2.

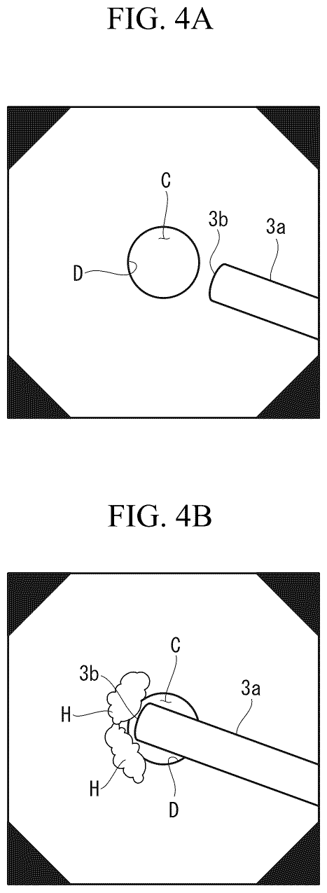

Specifically, the first operator causes the endoscope 2 to approach the target area C from an external naris G while observing an endoscopic image displayed on the display 6. As illustrated in FIG. 4A, the second operator, while observing the endoscopic image displayed on the display 6, causes the tube 3a of the spray device 3 to approach the target area C through the inside of the channel 2c of the endoscope 2, and positions the tip of the tube 3a in front of the opening D to the target area C.

Causing the spray device 3 to approach the target area C may be difficult in some cases due to the complex structure inside the nose and paranasal sinuses to be treated. In such cases, the following methods may be employed to curve the tip part of the spray device 3 in advance to correspond to the shape of the vicinity of the target area C.

A first method is to use a spray device 3 having a bending part and a bending control lever, similar to the endoscope 2. By operating the bending control lever, the bending part at the tip part of the spray device 3 can be bent.

A second method is to use a spray device 3 with plasticity in at least the tip part thereof. By applying force to the tip part of the spray device 3, the tip part of the spray device 3 can be curved at a desired angle allowing for easy insertion, and the shape curved at the desired angle can be maintained.

Next, in step S2, the second operator inserts the tip of the tube 3a into the target area C from the opening D while observing an endoscopic image including the opening D. If necessary, the first operator may insert the tip of the insertion part 2a into the target area C from the opening D.

Before step S2, the first or second operator may confirm the deposits and site of inflammation inside the target area C with the endoscope 2.

If the opening D is small, or if the opening D is obstructed by the mucosa or mucus, local anesthesia may be administered in the vicinity of the opening D and one of the following procedures may be performed.

A first procedure is to push and widen the opening D with the flexible endoscope 2, the spray device 3, an aspirating tube, or the like.

A second procedure is to aspirate the mucosa and mucus in the vicinity of the opening D with an aspirating tube.

A third procedure is to widen the opening D with a balloon.

A fourth procedure is to partially excise the mucosa or the opening D.

Steps S1 and S2 may be performed using a navigation system that displays the current position of the tip of the endoscope 2 or the spray device 3 inside the nose and paranasal sinuses on an image in real time. For example, the position of a marker attached to the tip of at least one of the endoscope 2 and the spray device 3 is detected magnetically, and the position of the marker is displayed on a previously taken CT image of the nose. The operators can easily insert the endoscope 2 and the spray device 3 up to the target area C on the basis of the position of the marker on the CT image.

Next, in step S3, the second operator operates the gas supply source 5, causing the air B to be supplied from the gas supply source 5 to the tube 3a, and the air B spurts out from the nozzle 3b inside the target area C. The pressure inside the target area C rises due to the supply of the air B, the air B spurts out from the gap around the spray device 3 at the opening D, and a strong flow of the air B proceeding toward the opening D is generated inside the target area C. Thus, as illustrated in FIG. 4B, the deposits H on the mucosa inside the target area C are pushed out of the target area C from the opening D by air pressure, and the deposits that had been presented on the mucosa are replaced with the air B. The pushed-out deposits H are expelled out of the nose and paranasal sinuses from the external naris G, internal nares, or the like.

Next, in step S4, the second operator operates the drug supply source 4, causing the drug A to be supplied from the drug supply source 4 to the tube 3a, and the drug A is sprayed from the nozzle 3b inside the target area C onto the mucosa. After spraying, the state of the target area C is observed with the endoscope 2, and step S4 is repeated if the drug A is not sprayed sufficiently in the target area C.

Next, in step S5, the second operator withdraws the tip of the tube 3a from the target area C by pulling on the tube 3a.

Next, in step S6, the second operator withdraws the tube 3a out of the nose and paranasal sinuses by pulling on the tube 3a.

Steps S5 and S6 may be performed while observing the interior of the nose and paranasal sinuses with the endoscope 2. Specifically, in step S5, the second operator withdraws the tube 3a from the target area C while observing an endoscopic image. Next, the first operator and the second operator pull on the insertion part 2a and the tube 3a, respectively, while observing an endoscopic image to back up the insertion part 2a and the tube 3a toward the external naris G at the same time.

In step S6, the spray device 3 may be withdrawn while spraying the drug A from the nozzle 3b by continuing to supply the drug A from the drug supply source 4 to the tube 3a. Rhinosinusitis often occurs not only in one location but in multiple locations simultaneously. By withdrawing the spray device 3 while spraying the drug A, the drug A can also be sprayed onto the mucosa over a wide area on the path of the spray device 3, and rhinosinusitis can be treated in multiple locations simultaneously.

In this way, according to the present embodiment, before the spraying of the drug A, the air B is sent into the target area C and the deposits are expelled out of the target area C by air pressure. Highly viscous and highly adherent deposits on the mucosa in the nasal cavity E or the paranasal sinuses F are difficult to wash away with a liquid such as a physiological saline solution, and are also difficult to aspirate. Such deposits can be expelled from the target area C effectively by air pressure, and the deposits on the mucosa can be replaced with the air B. Thus, the drug A can be brought into direct contact with the mucosa to obtain a high therapeutic effect of the drug A.

Furthermore, in the case of using a highly viscous drug A, the drug A can remain on the mucosa for a long time, thereby further improving the therapeutic effect of the drug.

Also, according to the present embodiment, in steps S1 and S2, the spray device 3 is inserted up to the target area C while observing the interior of the nose and paranasal sinuses with the endoscope 2. Thus, the spray device 3 can be made to reach the target area C along an appropriate path inside the complex nose and paranasal sinuses.

Also, according to the present embodiment, in step S4, by spraying the drug A from the tip of the tube 3a placed inside the target area C, the drug A can reach the deep part of the target area C, and the drug A can be applied with certainty over a wide area of the mucosa inside the target area C.

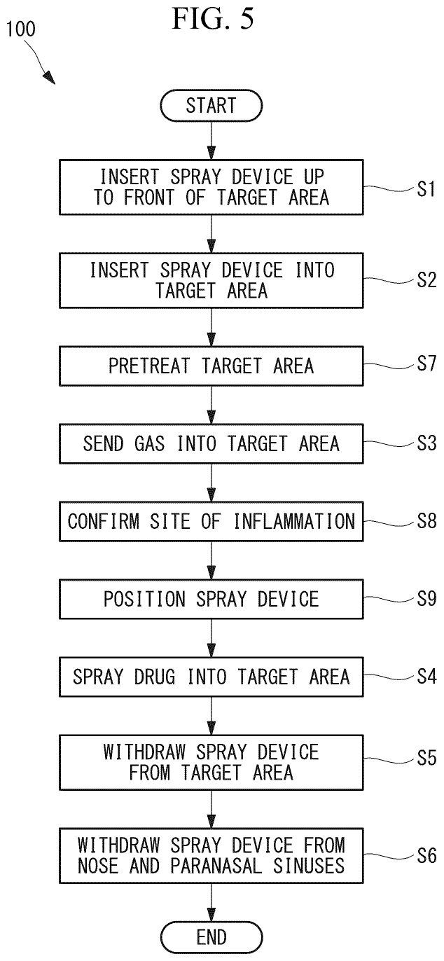

As illustrated in FIG. 5, the method 100 may further include at least one from among steps S7, S8, and S9.

Step S7 is a step for pretreating the target area C, to be performed before step S3. The timing of step S7 is chosen depending on the details of the pretreatment. Multiple types of pretreatment may be performed.

One example of pretreatment is to send a washing liquid such as a physiological saline solution into the target area C to wash the interior of the target area C. After step S2, the washing liquid may be sent into the target area C through the inside of the channel 2c or the tube 3a.

Another example of pretreatment is to aspirate deposits inside the target area C with an aspirating device. Before step S1, the aspirating device may be inserted into the nose and paranasal sinuses, and deposits inside the target area C may be expelled by aspiration.

By pretreatment such as washing or aspirating, components of deposits with low viscosity are expelled out of the target area C. This can heighten the effectiveness of expelling deposits with the air B to be performed later.

Step S8 is a step for confirming the site of inflammation inside the target area C with an endoscopic image, and step S9 is a step for positioning the spray device 3 with respect to the site of inflammation. Steps S8 and S9 are performed before step S4, being performed between steps S3 and S4, for example.

In step S8, the first operator observes the interior of the target area C through the opening D with an endoscopic image and confirms the site of inflammation. If the narrowness of the opening D makes it difficult to observe the interior of the target area C, the first operator may insert the tip of the endoscope 2 into the target area C from the opening D. After performing step S8, if the deposits H remain on the surface of the site of inflammation, the flow returns to step S3 to perform the operation for pushing the deposits H on the mucosa inside the target area C out of the target area C from the opening D by air pressure.

Next, in step S9, the second operator adjusts the position of the tip of the tube 3a while observing an endoscopic image, if necessary, so that the drug A will be sprayed toward the site of inflammation.

Thus, the drug A can be applied with greater certainty to the mucosa at the site of inflammation inside the target area C, and the therapeutic effect can be obtained with greater certainty.

Step S8 may be performed between steps S1 and S2 or between steps S2 and S3.

The endoscopic image that an operator observes in step S8 may be a normal endoscopic image or an endoscopic image in which the site of inflammation is displayed in an emphasized manner. The first operator can confirm the site of inflammation more easily on the basis of the emphasized area in the endoscopic image.

The image processor 7 may detect the site of inflammation in the endoscopic image with known technology such as image recognition, and cause the display 6 to display an endoscopic image in which the site of inflammation is displayed in an emphasized manner. The display 6 may be made to simultaneously display a normal endoscopic image and an endoscopic image in which the site of inflammation is emphasized. Otherwise, the display 6 may be made to alternatively display a normal endoscopic image and an endoscopic image in which the site of inflammation is emphasized, and an operator may toggle the image to be displayed on the display 6.

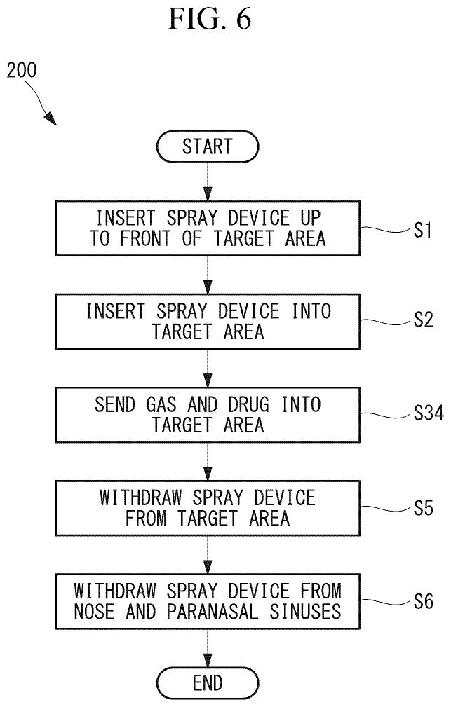

FIG. 6 illustrates a method 200 for treating rhinosinusitis according to a second embodiment. The method 200 includes, in place of steps S3 and S4, a step S34 for sending the air B and the drug A to the target area C simultaneously through the spray device 3. In step S34, the drug is sprayed onto the mucosa inside the target area C as deposits are expelled from the target area C by the air B. After step S34, the state of the target area C is observed with the endoscope 2, and step S34 is repeated if the drug A is not sprayed sufficiently in the target area C.

According to the method 200, the number of steps can be reduced and the procedure can be simplified. The method 200 may further include at least one from among steps S7, S8, and S9.

In the embodiments above, the first operator and the second operator operate the endoscope 2 and the spray device 3, respectively, but instead, a single operator may operate both the endoscope 2 and the spray device 3.

In the embodiments above, the air B is sent into the target area C in step S4, but a biocompatible gas other than the air B, such as an inert gas, for example, may be sent into the target area C.

In the embodiments above, the spray device 3 may also have a balloon at the tip part thereof. When the opening D is narrow and other cases, the balloon can be expanded at the opening D, if necessary, to widen the opening D. The balloon may be provided on the tip part of the endoscope 2.

In the embodiments above, the endoscope 2 is a flexible scope including the channel 2c. However, alternatively, the endoscope 2 may be a flexible scope or rigid scope not including the channel 2c. The rigid scope may be a forward-viewing scope or an oblique-viewing scope, and may include the bending part 2b. In this case, the spray device 3 is inserted into the target area C through the outside of the endoscope 2. A single operator may operate both the rigid scope and the spray device 3. Otherwise, a first operator may operate the rigid scope, and a second operator different from the first operator may operate the spray device 3.

In the embodiments above, the endoscope 2 and the spray device 3 are separate. However, alternatively, the endoscope and spray device may be unified. That is, an endoscope with the added function of a catheter or a spray device with the added function of a camera may be used.

Next, a method for treating rhinosinusitis according to a third embodiment of the present invention will be described with reference to the drawings.

The present embodiment is a method in which, in the treatment method according to the first embodiment, the spray device 3 is inserted up to the front of the target area C.

In the present embodiment, the image processor 7 can operate selectively in the two observation modes of a normal mode and a simulated color mode. The normal mode is an observation mode in which a white light image G1 acquired by the endoscope 2 is displayed on the display 6. The simulated color mode is an observation mode in which a discrimination image (simulated color image) G2 with a simulated color (discrimination color) applied to each pixel of the white light image G1 is generated and displayed on the display 6.

In the simulated color mode, a discrimination color is set for each pixel on the basis of an index Vl calculated by normalizing the sum of a red pixel value Vr and a green pixel value Vg of each pixel in the acquired white light image G1 by twice a blue pixel value Vb. The index may be “(Vr+Vg)/2Vb”, or if the calculation formula normalizes the sum of the red pixel value Vr and the green pixel value Vg by twice the blue pixel value Vb, the index may be calculated, for example, by the following calculation formula.

Vl=32×log 2((Vr+Vg)/2Vb)+256

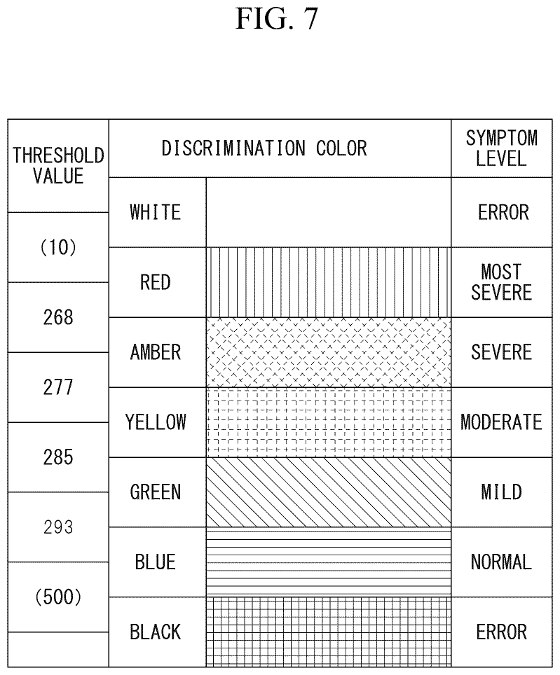

For example, as illustrated in FIG. 7, the method of setting a discrimination color on the basis of the index Vl may involve setting threshold values and discrimination colors for five-stage symptom levels “normal/mild/moderate/severe/most severe” and identifying the index Vl by threshold value to thereby assign a discrimination color to each symptom.

Discriminating among five-stage symptom levels requires four threshold values (a first threshold T1 to discriminate between normal and mild disease, a second threshold T2 to discriminate between mild and moderate disease, a third threshold T3 to discriminate between moderate and severe disease, and a fourth threshold T4 to discriminate between severe and most severe disease).

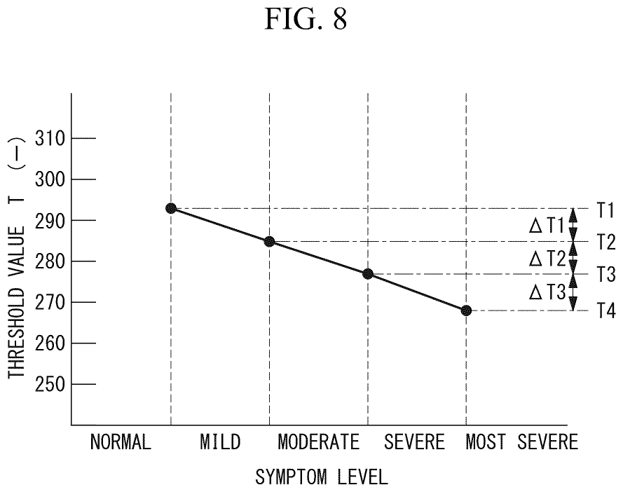

It is preferable for the threshold value differences ΔT1 to ΔT3, which illustrate an example of the threshold values T1 to T4 in FIG. 8, to be substantially the same. For example, the threshold value difference ΔT3 (threshold value T4−threshold value T3) is preferably equal to or greater than 80% and less than or equal to 120% of the threshold value difference ΔT2 (threshold value T3−threshold value T2). Depending on conditions, the threshold value difference ΔT1 may be greater than the threshold value difference ΔT2, and the threshold value difference ΔT2 may be greater than the threshold value difference ΔT3. Conversely, the threshold value difference ΔT1 may be less than the threshold value difference ΔT2, and the threshold value difference ΔT2 may be less than the threshold value difference ΔT3.

The observation mode can be switched manually by the first operator who operates the endoscope 2, for example. Also, a storage unit not illustrated can be connected to the image processor 7, and the discrimination image G2 generated in the simulated color mode can be recorded. The recording of the discrimination image G2 may also be directed manually by the first operator.

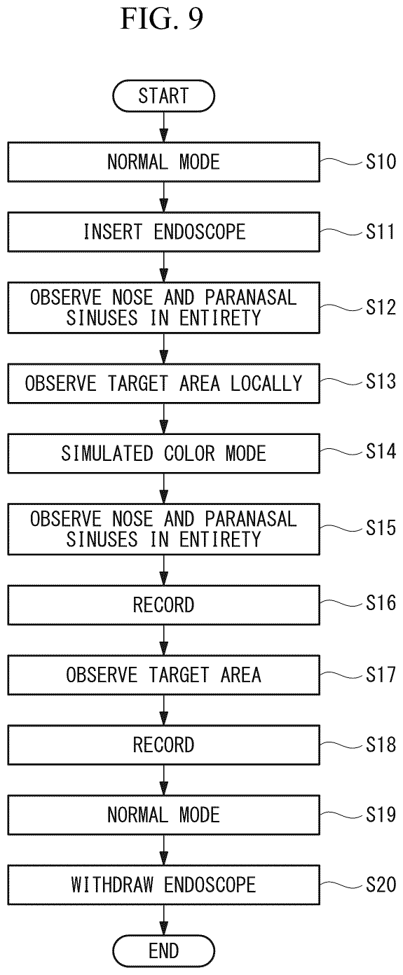

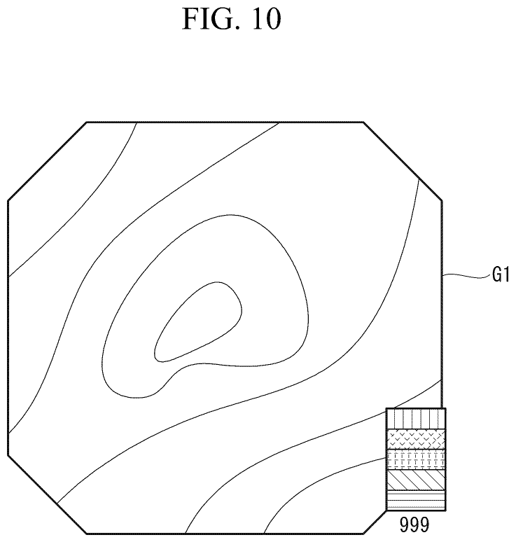

In the method according to the present embodiment, as illustrated in FIG. 9, first, the first operator switches the observation mode to the normal mode (step S10) and inserts the endoscope 2 into the nose and paranasal sinuses (step S11). The acquired white light image G1 is displayed on the display 6 as illustrated in FIG. 10, and the nose and paranasal sinuses in entirety are observed (step S12). Once the target area C of treatment inside the nose and paranasal sinuses is confirmed in the center on the display 6, the vicinity of the target area Cis displayed in enlarged manner to allow for local observation (step S13).

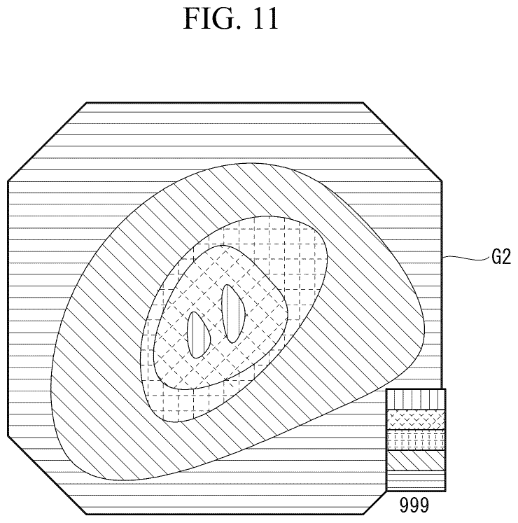

In this state, the first operator switches the observation mode from the normal mode to the simulated color mode (step S14). The simulated color image G2 is generated from the acquired white light image G1, displayed on the display 6 as illustrated in FIG. 11, and the nose and paranasal sinuses in entirety are observed (step S15). In the simulated color mode, the progression of a lesion in the nose and paranasal sinuses in entirety is differentiated into five-stage symptom levels and displayed, and thus the simulated color image G2 is recorded along with the five-stage symptom levels (step S16).

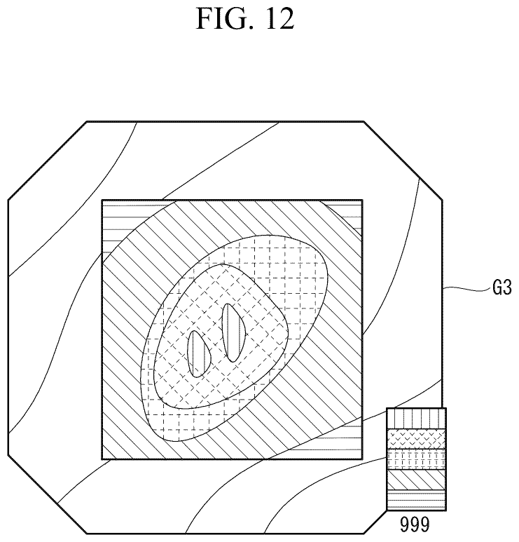

Next, a superimposed image G3, in which the region in the vicinity of the target area C in the center of the white light image G1 of the nose and paranasal sinuses in entirety acquired in the normal mode is replaced by the simulated color image G2 acquired in the simulated color mode, is displayed on the display 6 as illustrated in FIG. 12, and the target area C is observed (step S17). From the standpoint of visibility and operability, the area of the simulated color image G2 in the superimposed image is preferably equal to or greater than 20% and less than or equal to 70% of the area of the white light image G1 in entirety. The area of the simulated color image G2 in the superimposed image can be modified manually by an operator.

A superimposed image, in which the progression of a lesion in the target area C is differentiated into five-stage symptom levels, is recorded along with the five-stage symptom levels (step S18). Thereafter, the observation mode is switched to the normal mode (step S19) and the endoscope 2 is withdrawn from inside the nose and paranasal sinuses while the white light image G1 is displayed on the display 6 (step S20).

Before inserting the spray device 3 up to the front of the target area C, the white light image G1 and simulated color image G2 of the nose and paranasal sinuses in entirety, as well as the superimposed image G3 in which the central part of the white light image G1 is replaced by the simulated color image G2, are recorded along with the five-stage symptom levels, whereby the target area C to be sprayed with the drug A can be identified easily and treatment can be performed efficiently. Also, since the endoscope 2 is inserted and withdrawn while observing the white light image G1, the endoscope 2 can be inserted/withdrawn without damaging tissue inside the nose and paranasal sinuses.

Moreover, the recorded white light image G1, simulated color image G2, and superimposed image G3 of the nose and paranasal sinuses in entirety can be displayed along with the five-stage symptom levels, allowing a diagnosis result to be communicated to the patient in a manner that is easy to understand.

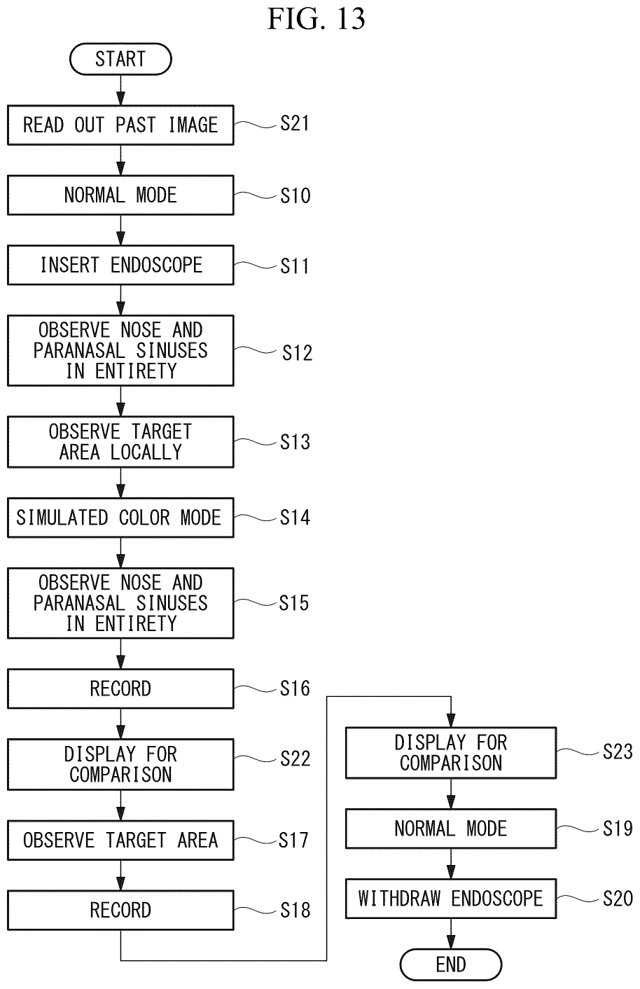

Note that in the present embodiment, since the white light image G1, the simulated color image G2, and the superimposed image G3 of the nose and paranasal sinuses in entirety are recorded along with the five-stage symptom levels, as illustrated in FIG. 13, a past image that has been recorded may also be read out before the insertion of the endoscope 2 (step S21). Additionally, after the recording of the simulated color image G2 of the nose and paranasal sinuses in entirety by the simulated color mode in step S16, the simulated color image G2 that was recorded and a simulated color image G2 that was read out in step S21 may be displayed in a comparable manner (side by side, for example) (step S22). Also, after the recording of the superimposed image G3 in step S18, the superimposed image G3 that was recorded and a superimposed image G3 that was read out in step S21 may be displayed in a comparable manner (side by side, for example) (step S23).

The recorded past and present simulated color images G2 and superimposed images G3 of the nose and paranasal sinuses in entirety can be presented to the patient in comparison with each other along with the five-stage symptom levels, allowing the effect of treatment to be communicated to the patient in a manner that is easier to understand.

Next, a method for treating rhinosinusitis according to a fourth embodiment of the present invention will be described with reference to the drawings.

In the description of the present embodiment, portions in common with the treatment method according to the second embodiment described above are denoted with the same signs, and a description of such portions is omitted.

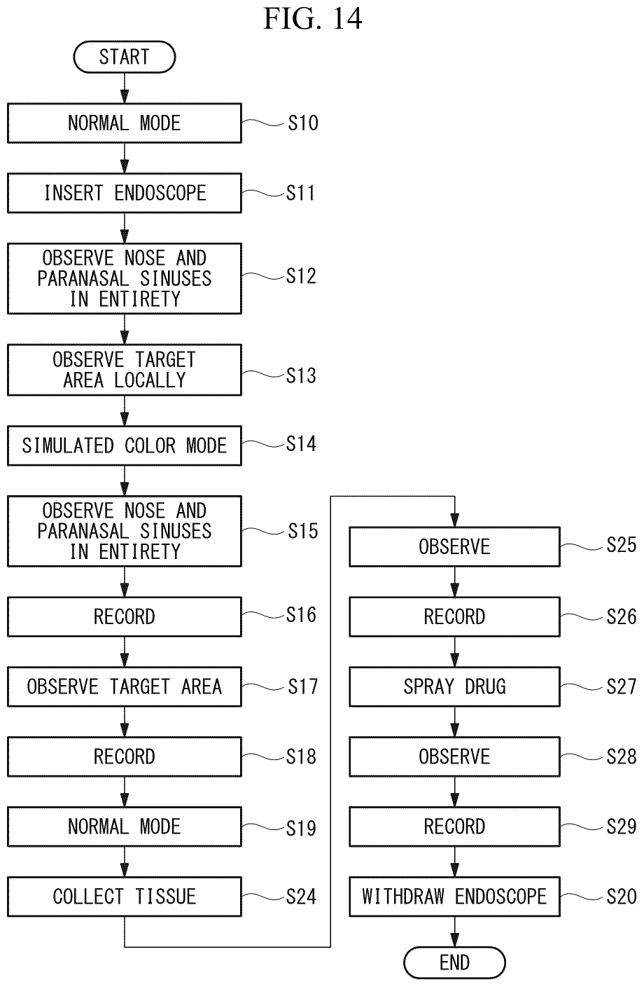

In the treatment method according to the present embodiment, as illustrated in FIG. 14, after the observation mode is switched to the normal mode in step S19 in the second embodiment, tissue collection (a biopsy) is performed while observing the white light image G1 displayed on the display 6 (step S24). After the tissue collection, the white light image G1 is displayed on the display 6 and observed (step S25), and the post-biopsy state is recorded (step S26).

Furthermore, in accordance with the first embodiment, drug spraying by the spray device 3 is performed (step S27), the white light image G1 is displayed on the display 6 and observed (step S28), the post-drug-spraying state is recorded (step S29), and the endoscope 2 is withdrawn (step S20). The observation according to steps S25 and S28 may also be performed with the simulated color image G2 acquired by switching the observation mode to the simulated color mode. In this case, it is necessary to switch the observation mode to the normal mode before the withdrawal of the endoscope 2 in step S20.

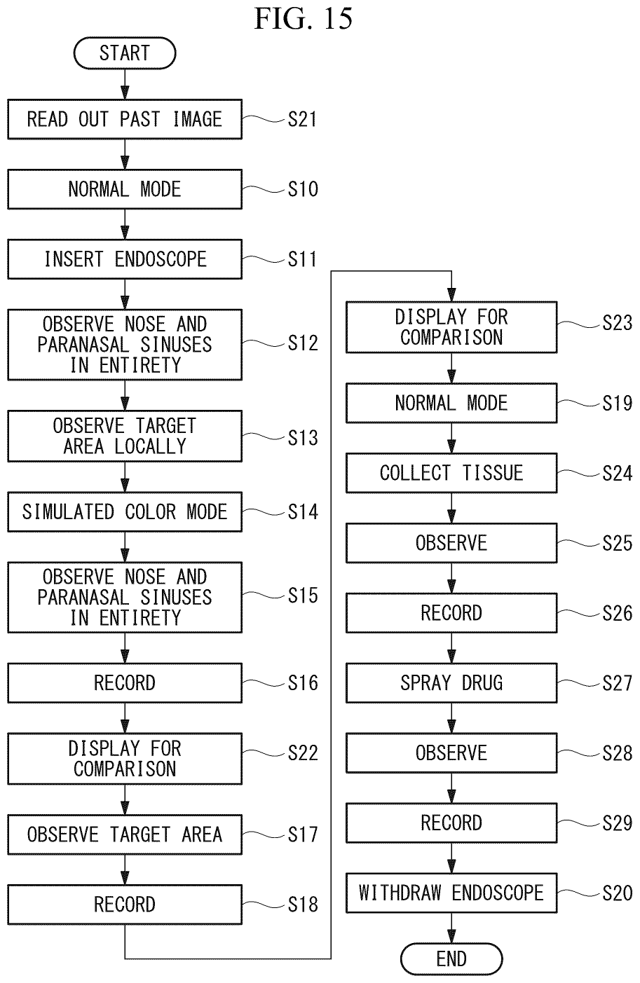

In this case, too, similarly to the second embodiment, a past image that has been recorded may also be read out before the insertion of the endoscope 2, as illustrated in FIG. 15 (step S21). Additionally, after the recording of the simulated color image G2 of the nose and paranasal sinuses in entirety by the simulated color mode in step S16, the simulated color image G2 that was recorded and a simulated color image G2 that was read out in step S21 may be displayed on the display 6 in a comparable manner (side by side, for example) (step S22). Also, after the recording of the superimposed image G3 in step S18, the superimposed image G3 that was recorded and a superimposed image G3 that was read out in step S21 may be displayed on the display 6 in a comparable manner (side by side, for example) (step S23).

The above describes embodiments and modifications of the present invention, but the present invention is not limited thereto, and the present invention may be altered, as appropriate, without departing from the scope thereof. For example, a method according to the present invention can be applied not only to rhinology but also the treatment of other body cavities, such as otology (auditory canal, middle ear, external auditory canal), respiratory organs (trachea, bronchus), GI (pancreaticobiliary ducts, diverticula), blood vessels, urethra, ureter, bladder, kidneys, uterus, vagina, fallopian tubes, and sperm ducts.

### REFERENCE SIGNS LIST

2 Endoscope
3 Spray device
100, 200 Method for treating rhinosinusitis
A Drug
B Air (gas)
C Target area
D Opening
E Nasal cavity
F Paranasal sinus
G1 White light image
G2 Discrimination image (simulated color image)
G3 Superimposed image
H Deposit

1. A method for treating rhinosinusitis, the method comprising:
inserting a tubular spray device into a target area of nose and paranasal sinuses;
sending a gas into the target area through the spray device to expel deposits from the target area; and
spraying a drug into the target area through the spray device.

2. The method according to claim 1, further comprising, before inserting the spray device into the target area,
inserting the spray device up to an opening to the target area or the vicinity thereof while observing the nose and paranasal sinuses with an endoscope.

3. The method according to claim 2, wherein the inserting the spray device into the target area is performed while observing the target area with the endoscope.

4. The method according to claim 2, wherein the spray device is inserted up to the opening to the target area or the vicinity thereof through a channel in the endoscope.

5. The method according to claim 1, further comprising, after spraying the drug,
withdrawing the spray device from the target area; and
withdrawing the spray device from the nose and paranasal sinuses.

6. The method according to claim 2, further comprising, before spraying the drug,
confirming a site of inflammation in the target area with an endoscopic image; and
positioning the spray device with respect to the site of inflammation.

7. The method according to claim 6, wherein the confirming the site of inflammation includes observing at least one of a normal endoscopic image and an endoscopic image in which the site of inflammation is emphasized.

8. The method according to claim 1, further comprising, before sending the gas, aspirating the deposits inside the target area with an aspirating device.

9. The method according to claim 2, wherein a first operator operates the endoscope, and a second operator different from the first operator operates the spray device.

10. A method for treating rhinosinusitis, the method comprising:
inserting a tubular spray device into a target area of nose and paranasal sinuses; and
sending a gas and a drug simultaneously into the target area through the spray device to spray the drug inside the target area while expelling deposits from the target area.

11. The method according to claim 1, further comprising, before inserting the spray device into the target area,
acquiring, with an image sensor, a white light image of a region including the target area;
calculating an index value for each pixel in at least a partial region of the acquired white light image by normalizing a sum of a red pixel value and a green pixel value of the each pixel by twice a blue pixel value
discriminating a symptom level of a lesion corresponding to the each pixel by using the index value recorded in advance and a threshold value indicating the symptom level;
generating a discrimination image by assigning a discrimination color corresponding to the discriminated symptom level to the each pixel; and
displaying the generated discrimination image.

12. The method according to claim 11, wherein the displaying the discrimination image includes displaying the symptom level for each the discrimination color in a confirmable way.

13. The method according to claim 11, wherein the generating the discrimination image includes generating the discrimination image with respect to the entire white light image of the region including the target area.

14. The method according to claim 11, wherein the generating the discrimination image includes generating a superimposed image in which the discrimination image of a partial region corresponding to the target area in the white light image is superimposed onto the white light image.

15. The method according to claim 11, further comprising:
recording the discrimination image.

16. The method according to claim 15, further comprising, after the recording the discrimination image,
collecting a portion of the target area while displaying the white light image; and
generating and recording the discrimination image by using the white light image acquired after the collecting, wherein
the spray device is inserted into the target area while displaying the white light image acquired afterward.

17. The method according to claim 15, further comprising:
reading out the discrimination image that was recorded in the past; and
displaying the past discrimination image that was read out and the newly generated discrimination image in a comparable manner.
